(19) United States
(12) Patent Application Publication
(10) Pub. No.: US 2004/0186112 A1
(43) Pub. Date: Sep. 23, 2004

(54) Polymorphic forms of dihydrochloride salts of cetirizine and processes for preparation thereof

(75) Inventors: Manne Satyanarayana Reddy, Hyderabad (IN); Thirumalai Rajan Srinivasan, Hyderabad (IN); Venkata Bhaskara Rao Uppala, Hyderabad (IN); Pattabhi Ramayya Vaddadi, Hyderabad (IN); Rajender Joga, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/729,856

(22) Filed: Dec. 4, 2003

(30) Foreign Application Priority Data
Dec. 4, 2002 (IN) 908/MAS/2002

(51) Int. Cl. A61K 31/495; C07D 241/04

(52) U.S. Cl. 514/255.04; 544/396

(57) ABSTRACT

The present invention relates to crystalline and amorphous forms of dextrorotatory dihydrochloride salt of cetirizine, the process for the preparation thereof and compositions containing the same. The invention also relates to the crystalline and amorphous forms of levorotatory dihydrochloride salt of cetirizine, the process for the preparation thereof and compositions containing the same. Both crystalline and amorphous salt forms of cetirizine dihydrochloride are suitable for pharmaceutical purposes in the treatment of allergies, including ailments such as chronic and acute allergic rhinitis, allergic conjunctivitis, pruritus, urticaria and the like.

### CROSS REFERENCE TO RELATED APPLICATIONS

This application claims benefit of a filing date of an Indian Patent Application No. 908/MAS/2002, filed Dec. 4, 2002, the contents of which are expressly incorporated herein by reference.

### FIELD OF THE INVENTION

The present invention relates to the crystalline form of dextrorotatory dihydrochloride salt of [2-[4-[(4-Chlorophenyl)-phenyl methyl]-1-piperazinyl] ethoxy] acetic acid [cetirizine], the process for the preparation of crystalline Form-1 dextrorotatory dihydrochloride salt of cetirizine, and compositions containing the same.

The present invention also relates to the crystalline form of levorotatory dihydrochloride salt of cetirizine, the process for preparation of crystalline Form-1 levorotatory dihydrochloride salt of cetirizine and compositions containing the same.

The present invention also relates to the amorphous form of dextrorotatory dihydrochloride salt of cetirizine, the process for preparation of the amorphous form of dextrorotatory dihydrochloride salt of cetirizine, and compositions containing the amorphous form of dextrorotatory dihydrochloride salt of cetirizine.

The present invention also relates to the amorphous form of levorotatory dihydrochloride salt of cetirizine, the process for preparation of the amorphous form of dextrorotatory dihydrochloride salt of cetirizine, and compositions containing the same.

### BACKGROUND OF THE INVENTION

Cetirizine and its salt, including its dihydrochloride, is known and is effective in the treatment of allergies, including but not limited to, chronic and acute allergic rhinitis, allergic conjunctivitis, pruritus, urticaria, and the like. Cetirizine belongs to the second generation of H₁ histamine receptor antagonists, which are believed to offer significant advantages over first generation compounds. Studies have shown that cetirizine provides safe and effective, symptomatic relief of seasonal allergies. Advantages include less sedation, low anticholinergic activity, and longer acting duration.

It is known that different polymorphic forms of the same drug may have substantial differences in certain pharmaceutically important properties. The amorphous form of a drug may exhibit different dissolution characteristics and in some cases different bioavailability patterns compared to crystalline forms. See, e.g., Konne T., Chem. Pharm. Bull. 38, 2003 (1990). For some therapeutic indications one bioavailability pattern may be favored over another. For example, the amorphous form of cefuroxime axetil exhibits higher bioavailability than its crystalline form. Further, amorphous and crystalline forms of a drug may have different handling properties, dissolution rates, solubility, and stability. For these reasons, among others, access to a choice between the amorphous or crystalline form of drug is desirable for different applications. Therefore, there is a need for new solid forms of cetirizine dihydrochloride and new methods of preparation.

### SUMMARY OF INVENTION

In accordance with one aspect, the present invention provides a new crystalline Form I dextrorotatory dihydrochloride salt of cetirizine. Preferably, the crystalline Form-I dextrorotatory dihydrochloride salt of cetirizine has an X-ray diffraction pattern that includes five or more peaks selected from the group consisting of peaks with 2 theta angles of 7.05±0.09, 7.96±0.09, 14.35±0.09, 14.81±0.09, 17.39±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09 and 30.57±0.09. More preferably, crystalline Form-I dextrorotatory dihydrochloride salt of cetirizine has substantially the same X-ray diffraction pattern as shown in FIG. 1.

In accordance with another aspect, the invention provides a pharmaceutical composition that includes a prophylactically or therapeutically effective amount of the crystalline Form-I dextrorotatory dihydrochloride salt of cetirizine and one or more pharmaceutically acceptable excipients. Preferably, crystalline Form-I dextrorotatory dihydrochloride salt of cetirizine has an X-ray diffraction pattern which includes five or more peaks selected from the group consisting of peaks with 2 theta angles of 7.05±0.09, 7.96±0.09, 14.35±0.09, 14.81±0.09, 17.39±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09 and 30.57±0.09.

In accordance with yet another aspect, the invention provides a process for preparation of the crystalline Form-I of dextrorotatory dihydrochloride salt of cetirizine that includes a) providing a solution of 2-[2-[4-[(4-Chlorophenyl)-phenyl methyl]-1-piperazinyl] ethoxy] acetic acid as in a ketone containing solvent; b) treating the solution with hydrochloric acid, wherein the hydrochloric acid is present in an amount sufficient to form a di-hydrochloric acid salt of 2-[2-[4-[(4-Chlorophenyl)-phenyl methyl]-1-piperazinyl] ethoxy] acetic acid which separates as a solid mass; and c) isolating the solid mass to obtain the crystalline Form-I dextrorotatory dihydrochloride salt of cetirizine. Pharmaceutical compositions that include a prophylactically or therapeutically effective amount of crystalline Form-I dextrorotatory dihydrochloride salt of cetirizine produced by the process described, and one or more pharmaceutically acceptable excipients are also provided.

In accordance with yet another aspect, the invention provides a new crystalline Form-I levorotatory dihydrochloride salt of cetirizine has an X-ray diffraction pattern that includes five or more peaks selected from the group consisting of peaks with 2 theta angles of: 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09. More preferably, crystalline Form-I levorotatory dihydrochloride salt of cetirizine has substantially the same X-ray diffraction pattern as shown in FIG. 2.

In accordance with another aspect, the invention provides a pharmaceutical composition that includes a prophylactically or therapeutically effective amount of the crystalline Form-I levorotatory dihydrochloride salt of cetirizine and one or more pharmaceutically acceptable excipients. Preferably, crystalline Form-I levorotatory dihydrochloride salt of cetirizine has an X-ray diffraction pattern with the following peaks: 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

In accordance with yet another aspect, the invention provides a process for preparation of the crystalline Form-I of levorotatory dihydrochloride salt of cetirizine that includes a) providing a solution of 2-[2-[4-[(4-Chlorophenyl)-phenyl methyl]-1-piperazinyl] ethoxy] acetic acid in a ketone containing solvent; b) treating the solution with hydrochloric acid, wherein the hydrochloric acid is present in an amount sufficient to form a di-hydrochloric acid salt of 2-[2-[4-[(4-Chlorophenyl)-phenyl methyl]-1-piperazinyl] ethoxy] acetic acid which separates as a solid mass; and c) removing the volatile components of the solvent thereby a solid separates and d) isolating the solid mass to obtain the crystalline Form-I levorotatory dihydrochloride salt of cetirizine. Pharmaceutical compositions that include a prophylactically or therapeutically effective amount of crystalline Form-I levorotatory dihydrochloride salt of cetirizine produced by the process described, and one or more pharmaceutically acceptable excipients are also provided.

In accordance with another aspect, the present invention provides an amorphous form of dextrorotatory dihydrochloride salt of cetirizine. In accordance with another aspect, the invention provides a pharmaceutical composition that includes a prophylactically or therapeutically effective amount of an amorphous form of dextrorotatory dihydrochloride salt of cetirizine that is substantially free of its crystalline form and one or more pharmaceutically acceptable excipients. The pharmaceutical compositions of this aspect of the invention may be formulated, for example, as solid dosage forms for oral administration. In accordance with yet another aspect, the invention provides a composition containing a solid form of dextrorotatory dihydrochloride salt of cetirizine, which is at least 80% amorphous.

In accordance with yet another aspect, the invention provides a process for preparation of an amorphous form of dextrorotatory dihydrochloride salt of cetirizine. In one embodiment of this aspect of the invention, the process involves dissolution of cetirizine salt or free base in an aqueous mixture of water immiscible solvent using hydrochloric acid and further isolation by adding a water immiscible aliphatic hydrocarbon solvent. Pharmaceutical compositions that include a prophylactically or therapeutically effective amount of the amorphous form of dextrorotatory dihydrochloride salt of cetirizine produced by the process described, and one or more pharmaceutically acceptable excipients are also provided.

In accordance with another aspect, the present invention provides an amorphous form of levorotatory dihydrochloride salt of cetirizine. In accordance with another aspect, the invention provides a pharmaceutical composition that includes a prophylactically or therapeutically effective amount of an amorphous form of levorotatory dihydrochloride salt of cetirizine that is substantially free of its crystalline form and one or more pharmaceutically acceptable excipients. The pharmaceutical compositions of this aspect of the invention may be formulated, for example, as solid dosage forms for oral administration. In accordance with yet another aspect, the invention provides a composition containing a solid form of levorotatory dihydrochloride salt of cetirizine, which is at least 80% amorphous.

In accordance with yet another aspect, the invention provides a process for preparation of an amorphous form of levorotatory dihydrochloride salt of cetirizine. In one embodiment of this aspect of the invention, the process involves dissolution of cetirizine salt or free base in an aqueous mixture of water immiscible solvent using hydrochloric acid and further isolation by adding a water immiscible aliphatic hydrocarbon solvent. Pharmaceutical compositions that include a prophylactically or therapeutically effective amount of the amorphous form of levorotatory dihydrochloride salt of cetirizine produced by the process described, and one or more pharmaceutically acceptable excipients are also provided.

The processes described herein are believed to be simple, eco-friendly and cost-effective. The pharmaceutical compositions of this invention may be formulated, for example, as solid dosage forms for oral administration.

### BRIEF DESCRIPTION OF THE ACCOMPANYING DRAWINGS

FIG. 1 is a diagram showing an X-ray powder diffraction pattern of crystalline Form-I dextrorotatory dihydrochloride salt of cetirizine.

FIG. 2 shows the X-ray powder diffraction pattern of crystalline Form-I Levorotatory dihydrochloride salt of cetirizine.

FIG. 3 shows the X-ray powder diffraction pattern of an amorphous form of dextrorotatory dihydrochloride salt of cetirizine.

FIG. 4 shows the X-ray powder diffraction pattern of an amorphous form of levorotatory dihydrochloride salt of cetirizine.

FIG. 5 is a differential scanning colorimetry thermogram of crystalline Form-I dihydrochloride salt of cetirizine.

FIG. 6 is an Infrared spectrum of crystalline Form-I dihydrochloride salt of cetirizine.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

The crystalline compounds designated herein as “crystalline Form I dextrorotatory dihydrochloride salt of cetirizine” and “crystalline Form I levorotatory dihydrochloride salt of cetirizine” are new polymorphs of cetirizine dihydrochloride that are different from known polymorphs. Both can be characterized via X-ray powder diffraction and are further described below.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes but is not limited to a solution, a suspension, a gel, an ointment, an emulsion and/or mixtures thereof. The term composition is intended to encompass a product comprising the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing one of the dihydrochloride salts of cetirizine described by the present invention, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevent the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or desired product. It should be appreciated that the reaction which produces the indicated and/or desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art. When a composition is said to be “substantially free of” a substance, this means that said substance in said composition is present in such a low amount as not be detectable in said composition, or it means that said substance is absent from said composition.

Cetirizine dihydrochloride is a compound of the formula:

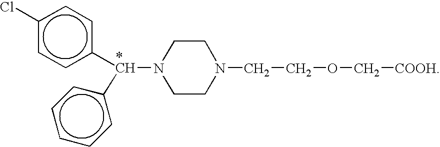

The R enantiomer is referred to as levocetirizine and the S enantiomer is referred to as dextrocetirizine. As used herein, “cetirizine” is a generic term that denotes the racemic mixture of R and S enantiomers (with respect to the asymmetric center marked with the asterisk) as well as each of the enantiomers separately. Thus, the term “substantially free of crystalline forms of cetirizine dihydrochloride,” as used herein, means that the crystalline form of cetirizine dihydrochloride cannot be detected by methods known to those skilled in the art.

The process for the preparation of levocetirizine and its salt including dihydrochloride is known. For example, GB 2 225 321 A discloses a process for preparation of levocetirizine and its dihydrochloride, which includes treating cetirizine with an acid or a base in an aqueous, alcoholic or aqueous-alcoholic medium, which is then subjected to hydrolysis and converted into levocetirizine or its dihydrochloride. The portions of the '321 patent and its U.S. counterparts, if any, which show the preparation process is/are incorporated herein by reference. An article in Tetrahedron Letters 37(28), 4837-4840 (1996), which is incorporated herein by reference, discloses the enantioselective synthesis of levocetirizine dihydrochloride and its further purification via ion exchange chromatography.

Different solid forms of the same drug may exhibit different properties, including characteristics that have functional implications with respect to their use as active ingredients of pharmaceutical products. For example, polymorphs of the same drug may have substantial differences in such pharmaceutically important properties as dissolution rates and bioavailability. Likewise, different polymorphs may have different processing properties, such as hydroscopicity, flowability, and the like, which could affect their suitability as active pharmaceuticals for commercial production.

According to one aspect, the invention provides a crystalline form of dextrorotatory dihydrochloride salt of cetirizine. The specific crystalline form obtained by the inventors is designated as Form-I. Likewise, the invention also provides a crystalline form of levorotatory dihydrochloride salt of cetirizine. The crystalline Form I dextrorotatory dihydrochloride salt of cetirizine may be prepared, for example, by converting a salt of cetirizine to the dihydrochloride with in situ crystallization. For example, the process may involve providing a solution of a salt of cetirizine in an organic solvent; adding alcoholic hydrochloric acid solution; stirring the solution until separation of a solid mass of cetirizine dihydrochloride; and isolating and drying the product. Ester solvents, such as methyl acetate, ethyl acetate, tertiary butyl acetate, isopropyl acetate, isobutyl acetate and mixture thereof, are preferred for dissolving the starting cetirizine, while the preferred solvent carrier for hydrochloric acid is isopropanol. Preferably, the resulting crystalline cetirizine dihydrochloride is dried at a temperature of from about 40° C. to about 100° C. The chemical synthesis of starting salt cetirizine may be affected by any method known in the art. For example, the synthesis described in U.S. Pat. No. 4,525,358 cited above and incorporated by reference herein in its entirety, may be used for this purpose.

Both the crystalline Form I dextrorotatory dihydrochloride salt of cetirizine and Form I levorotatory dihydrochloride salt of cetirizine may be characterized by X-ray diffraction. X-ray diffraction patterns are unique for different crystalline forms. Each crystalline form exhibits a diffraction pattern with a unique set of diffraction peaks that can be expressed in 2 theta angles, d-spacing values and relative peak intensities. 2 theta diffraction angles and corresponding d-spacing values account for positions of various peaks in the X-ray powder diffraction pattern. D-spacing values are calculated with observed 2 theta angles and copper K(α1) wavelength using the Bragg equation well known to those of skill in the art.

However, slight variations in observed 2 theta angles or d-spacing values are expected based on the specific diffractometer employed the analyst and the sample preparation technique. More variation is expected for the relative peak intensities. Identification of the exact crystal form of a compound should be based primarily on observed 2 theta angles with lesser importance attributed to relative peak intensities.

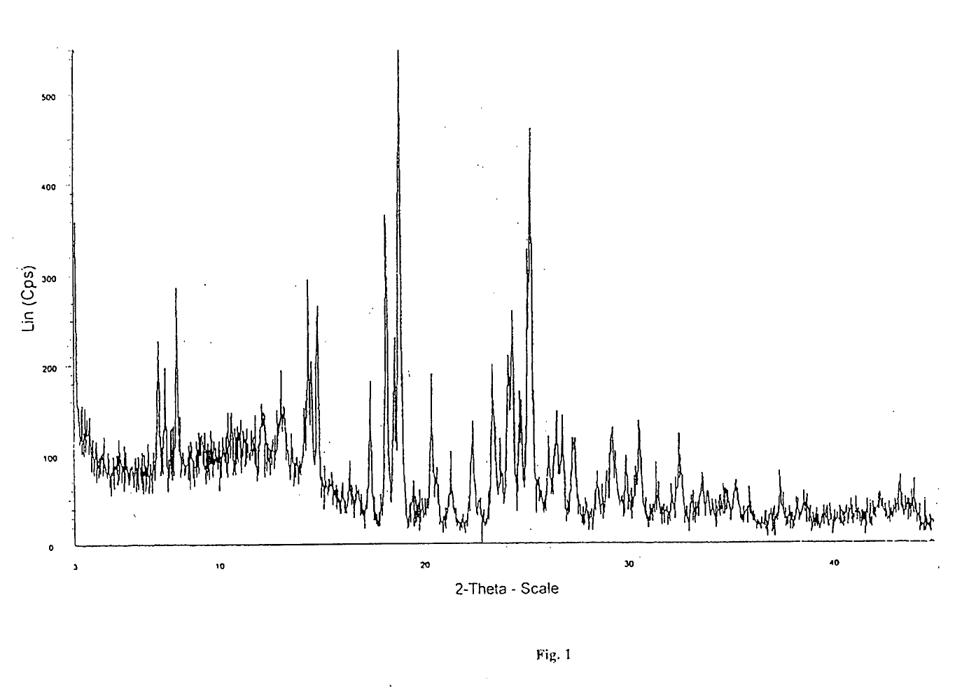

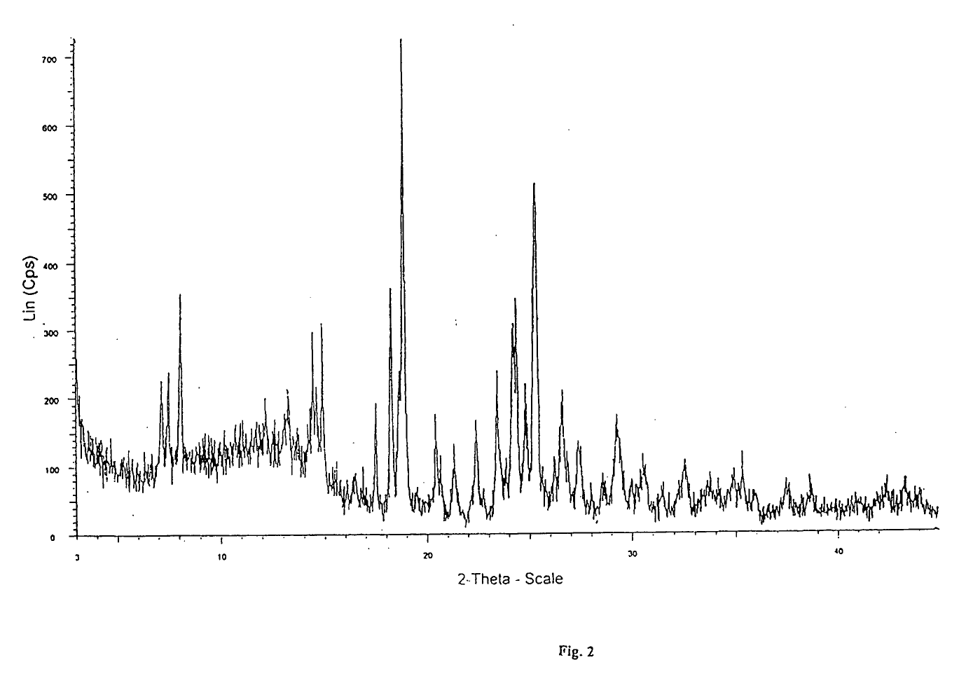

FIG. 1 shows an X-ray powder diffraction pattern of the crystalline Form I dextrorotatory dihydrochloride salt of cetirizine. FIG. 2 shows an X-ray powder diffraction pattern of crystalline Form I levorotatory dihydrochloride salt of cetirizine. Both X-ray powder diffraction patterns were obtained on a Bruker Axs, D8 Advance Powder X-ray Diffractometer with Cu K alpha-1 Radiation source.

Some margin of error is present in each of the 2 theta angle assignments and d-spacings reported herein. The assigned margin of error in the 2 theta angles for Form I dextrorotatory dihydrochloride salt of cetirizine and Form I levorotatory dihydrochloride salt of cetirizine is approximately ±0.09 for each of the peak assignments. In view of the assigned margin of error, the crystalline Form-I dextrorotatory dihydrochloride salt of cetirizine of the invention may be characterized by an X-ray powder diffraction pattern that includes five or more peaks selected from the group consisting of peaks with 2 theta angles of 7.05±0.09, 7.96±0.09, 14.35±0.09, 14.81±0.09, 17.40±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09 and 30.57±0.09. The crystalline Form-I levorotatory dihydrochloride salt of cetirizine of the invention may be characterized by an X-ray powder diffraction pattern that includes five or more peaks selected from the group consisting of peaks with 2 theta angles of 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline salt form of cetirizine dihydrochloride obtained using the methods described herein, over FIG. 1 and readily determine whether the X-ray diffraction pattern of the unidentified form is substantially the same as the X-ray powder diffraction pattern of Form I. If the X-ray powder diffraction pattern is substantially the same as FIG. 1, the previously unknown crystalline form can be readily and accurately identified as Form I crystalline dextrorotatory dihydrochloride salt of cetirizine.

Although 2 theta angles or d-spacing values are the primary methods of identifying the crystalline form, it may be desirable to also compare relative peak intensities. As noted above, relative peak intensities may vary depending upon the specific diffractometer employed and the analyst's sample preparation technique. The peak intensities are reported as intensities relative to the peak intensity of the strongest peak.

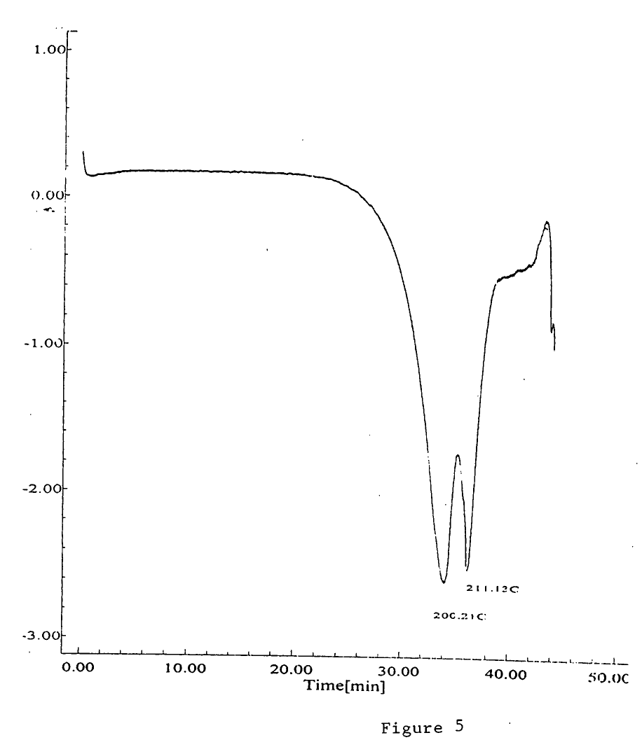

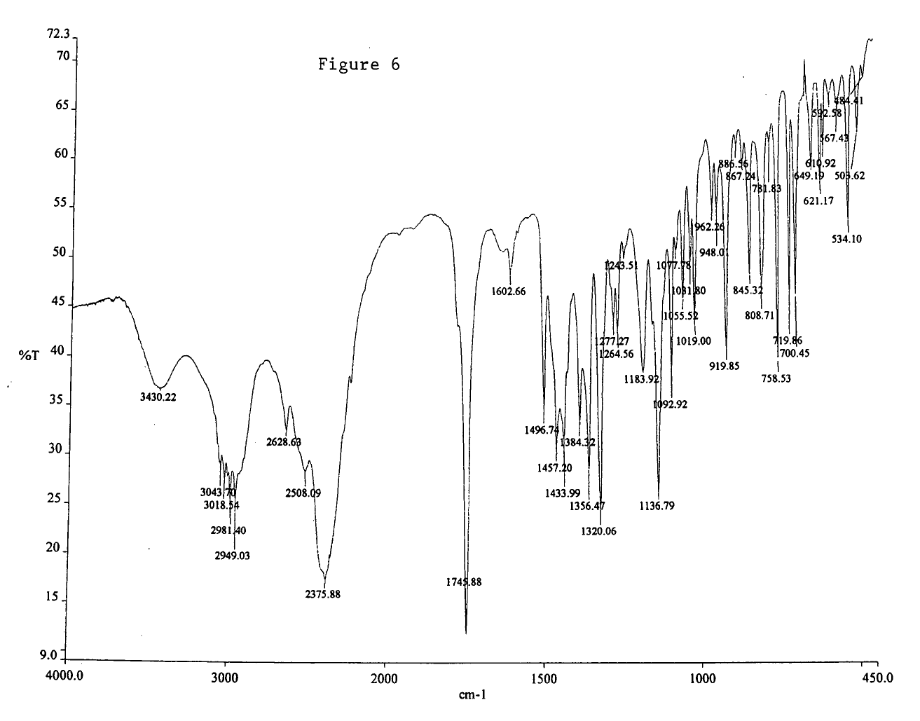

The crystalline form of the dihydrochloride salt of cetirizine may be also characterized by differential scanning calorimetry and/or infrared spectroscopy. The DSC thermogram of crystalline Form I of cetirizine dihydrochloride salt obtained by the inventors is shown in FIG. 5. It exhibits a significant endo-endo pattern with identified peaks around 195° C. and 215° C. It was measured on Schimadzu differential scanning colorimeter in a temperature range of 50-250° C. with a heating rate of 5° C./minute. The infrared spectrum of crystalline Form I of dihydrochloride salt of cetirizine obtained by the inventors is shown in FIG. 6. It was measured on Perkin-Elmer FT-IR instrument by KBr-transmission method. The significant bands may be identified at approximately 3430.22, 2949.03, 2375.88, 1745.88, 1496.74, 1496.74, 1320.06, 1136.79, 919.85, 758.53, 719.86, 700.45 and 534.10 cm⁻¹.

The present invention also provides the amorphous forms of both the dextrorotatory dihydrochloride salt of cetirizine and levorotatory dihydrochloride salt of cetirizine. The processes for preparing the amorphous forms are also provided. The inventors concluded that amorphous, free-flowing forms of cetirizine dihydrochloride salt are useful in pharmaceutical applications because, among other reasons, they can be easily handled in pharmaceutical processing. Advantages to using the amorphous forms of the dihydrochloride salts of cetirizine also include enhanced solubility.

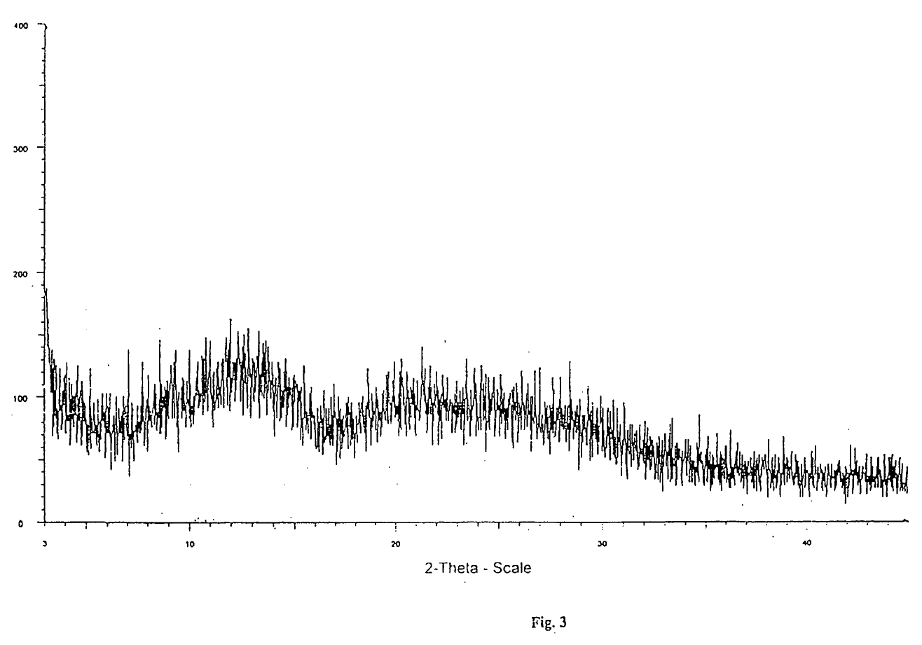

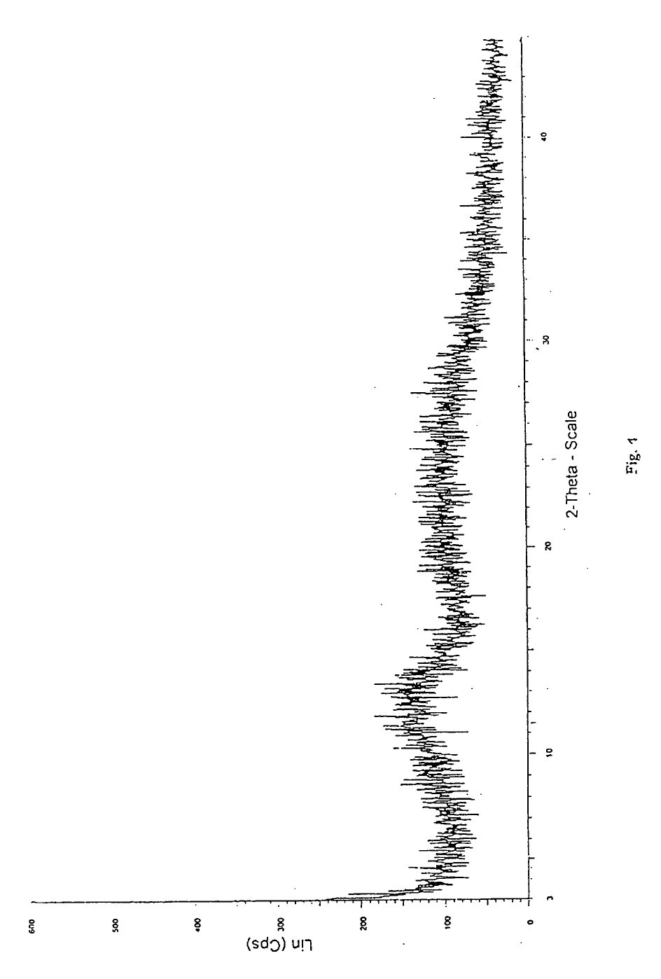

FIG. 3 shows the X-ray powder diffraction of amorphous form of dextrorotatory dihydrochloride salt of cetirizine. FIG. 4 shows the X-ray powder diffraction of amorphous form of levorotatory dihydrochloride salt of cetirizine. The X-ray powder diffraction patterns of the amorphous forms of cetirizine dihydrochloride were measured on a Bruker Axs, D8 Advance Powder X-ray Diffractometer with Cu K alpha-1 Radiation source.

The invention also provides for compositions containing dextrorotatory dihydrochloride salt of cetirizine which are at least 80% amorphous, by total weight of dextrorotatory dihydrochloride salt of cetirizine in the composition. The remainder of dextrorotatory dihydrochloride salt of cetirizine in the composition, i.e., 20% or less of the total weight of dextrorotatory dihydrochloride salt of cetirizine may be, for example, a crystalline form of cetirizine dihydrochloride. In a more preferred embodiment, the composition contains at least 90% of the amorphous form with respect to total weight of dextrorotatory dihydrochloride salt of cetirizine in the composition. Yet more preferably, the composition contains at least 95% of the amorphous form with respect to total weight of dextrorotatory dihydrochloride salt of cetirizine in the composition. In the most preferred embodiment, the composition is substantially free of crystalline forms of cetirizine dihydrochloride.

In one preferred variant, the composition includes at least a small amount of crystalline cetirizine dihydrochloride, preferably, crystalline Form I dextrorotatory dihydrochloride salt of cetirizine. In a non-limiting example, the composition includes at least 80% of amorphous dextrorotatory cetirizine dihydrochloride and at least 1% crystalline cetirizine dihydrochloride. In another non-limiting example, the composition includes at least 80% of amorphous dextrorotatory cetirizine dihydrochloride and at least 5% crystalline cetirizine dihydrochloride. All compositions, in 0.1% increments, which include at least 80% of amorphous dextrorotatory cetirizine dihydrochloride and at least 1% crystalline cetirizine dihydrochloride are contemplated. All percentages are based upon the total amount of dextrorotatory cetirizine dihydrochloride in the composition.

The invention also provides for compositions containing levorotatory dihydrochloride salt of cetirizine which are at least 80% amorphous, by total weight of levorotatory dihydrochloride salt of cetirizine in the composition. The remainder of levorotatory dihydrochloride salt of cetirizine in the composition, i.e., 20% or less of the total weight of levorotatory dihydrochloride salt of cetirizine may be, for example, a crystalline form of cetirizine dihydrochloride. In a more preferred embodiment, the composition contains at least 90% of the amorphous form with respect to total weight of levorotatory dihydrochloride salt of cetirizine in the composition. Yet more preferably, the composition contains at least 95% of the amorphous form with respect to total weight of levorotatory dihydrochloride salt of cetirizine in the composition. In the most preferred embodiment, the composition is substantially free of crystalline forms of cetirizine dihydrochloride.

In one preferred variant, the composition includes at least a small amount of crystalline cetirizine dihydrochloride, preferably, crystalline Form I levorotatory dihydrochloride salt of cetirizine. In a non-limiting example, the composition includes at least 80% of amorphous levorotatory cetirizine dihydrochloride and at least 1% crystalline cetirizine dihydrochloride. In another non-limiting example, the composition includes at least 80% of amorphous levorotatory cetirizine dihydrochloride and at least 5% crystalline cetirizine dihydrochloride. All compositions, in 0.1% increments, which include at least 80% of amorphous levorotatory cetirizine dihydrochloride and at least 1% crystalline cetirizine dihydrochloride are contemplated. All percentages are based upon the total amount of levorotatory cetirizine dihydrochloride in the composition.

X-ray diffraction provides a convenient and practical means for quantitative determination of the relative amounts of crystalline and amorphous forms. The X-ray powder diffraction method is capable of providing both qualitative and quantitative information about compounds present in a solid sample. X-ray diffraction is adaptable to quantitative applications because the intensities of the diffraction peaks of a given compound in a mixture are proportional to the faction of the material in the mixture.

The identification of a form of a compound from its powder diffraction pattern is based upon the position of the lines in terms of theta and their relative intensities. The diffraction angle 2 theta is determined by the spacing between a particular set of planes. Using the Bragg equation, the distance d is readily calculated from the known wavelength of the source and the measured angle.

Identification of the crystalline form is empirical. By measuring the intensity of the diffraction lines and comparing them with standards, it is possible to make a quantitative analysis of crystalline mixtures. Qualitative information can be converted to quantitative data by measuring the peak heights. Two methods that are used to analyze X-ray diffraction quantitatively are the Internal Standard Method and the External Standard Method. The Internal Standard Method is the preferred procedure for analyzing powdered systems. This method measures a known quantity of a reference powder which is added to an unknown powder. The mass absorption coefficient of the mixture need not be known in advance. Any number of constituents in the mixture may be quantified independently, including the amorphous (non-crystalline) components. The External Standard Method is used to analyze solid systems when the mass absorption co-efficient is known. It allows the quantification of one or more components in a system, which may contain an amorphous fraction.

Crystalline content may be characterized by X-ray diffraction. The X-ray diffraction pattern for the crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(α1) wavelength using the Bragg equation. Slight variations in observed 2 theta angles or d-spacing values are expected based on the specific diffractometer employed the analyst and the sample preparation technique. More variation is expected for the relative peak intensities. Identification of the crystal form of a compound should be based primarily on observed 2 theta angles with lesser importance attributed to relative peak intensities.

The amorphous form of dextrorotatory cetirizine dihydrochloride of the present invention has an X-ray powder diffractogram pattern substantially as depicted in FIG. (3). The amorphous form of levorotatory cetirizine dihydrochloride of the present invention has an X-ray powder diffractogram pattern substantially as depicted in FIG. (4). The X-ray powder diffraction pattern shows no peaks and gave a plain halo, thus demonstrating the-amorphous nature of the product. All diffractograms were obtained on a Bruker Axs, D8 Advance Powder X-ray Diffractometer with Cu K alpha-1 Radiation source. Table 1 below shows 2 theta and intensity values, as measured by the inventors, for the crystalline forms of cetirizine dihydrochloride and its individual enantiomers:

TABLE 1

| Dextrorotatory | | Levorotatory | | | |
|---|---|---|---|---|---|
| dihydrochloride salt of | | dihydrochloride salt of | | | |
| cetirizine | | cetirizine | | Cetirizine | |
| 2 theta | | 2 theta | | dihydrochloride | |
| (°) | Intensity(%) | (°) | Intensity(%) | 2-Theta(°) | Intensity(%) |
| 18.815 | 100 | 18.855 | 100 | 18.637 | 100.0 |
| 25.247 | 73.2 | 25.311 | 79.2 | 18.244 | 81.1 |
| 18.170 | 59.5 | 18.244 | 48.9 | 25.115 | 78.8 |
| 14.805 | 35.6 | 24.211 | 41.0 | 14.423 | 47.9 |
| 24.325 | 34.6 | 24.361 | 40.5 | 17.328 | 35.9 |
| 18.591 | 29.9 | 8.018 | 37.2 | 8.007 | 28.0 |
| 14.347 | 29.0 | 14.87 | 34.2 | 20.388 | 27.8 |
| 24.158 | 28.2 | 18.648 | 30.8 | 24.143 | 25.8 |
| 7.955 | 27.1 | 23.415 | 27.5 | 7.099 | 25.4 |
| 23.354 | 27.0 | 14.408 | 26.1 | 14.731 | 22.5 |
| 17.394 | 23.4 | 26.602 | 24.7 | 23.432 | 20.7 |
| 7.053 | 23.2 | 22.388 | 21.6 | 12.966 | 20.9 |
| 20.327 | 21.7 | 17.475 | 20.6 | 22.949 | 17.8 |
| 22.330 | 19.5 | 7.096 | 19.7 | 26.109 | 16.5 |
| 24.727 | 19.0 | 24.812 | 19.5 | 29.204 | 11.3 |
| 27.347 | 17.7 | 29.282 | 19.1 | 26.706 | 10.7 |
| 30.571 | 16.8 | 7.424 | 18.8 | 8.756 | 9.9 |
| 26.514 | 16.5 | 20.42 | 18.7 | 19.965 | 9.0 |
| 26.799 | 16.3 | 27.385 | 16.1 | 15.923 | 8.8 |

The percent composition of crystalline dextrorotatory cetirizine dihydrochloride salt can be determined in an unknown composition. The X-ray powder diffraction patterns of an unknown composition can be compared to a known standard containing pure crystalline dextrorotatory cetirizine dihydrochloride salt to identify the percent ratio of the crystalline form of dextrorotatory cetirizine dihydrochloride salt. This is done by comparing the relative intensities of the peaks from the diffraction pattern of the unknown composition with a calibration curve derived from the X-ray diffraction pattern of a pure crystalline sample of dextrorotatory cetirizine dihydrochloride salt. The curve can be calibrated based on the X-ray powder diffraction pattern for the strongest peak from a pure sample of crystalline dextrorotatory cetirizine. The peak intensities are reported as intensities relative to the peak intensity of the strongest peak (“the 100% peak”). Likewise, the percent composition of levorotatory cetirizine dihydrochloride salt can be identified in the same manner. The 100% peak for cetirizine dihydrochloride is at 2-theta ˜18.64, for levorotatory dihydrochloride salt of cetirizine it is at ˜18.85, and for dextrorotatory dihydrochloride salt of cetirizine it is at ˜18.81 (TABLE 1).

The calibration curve may be created in a manner known to those of skill in the art. For example, five or more artificial mixtures of amorphous and crystalline salts of cetirizine dihydrochloride, at different amounts, may be prepared. In a non-limiting example, such mixtures may contain, 2%, 5%, 7%, 8%, and 10% of crystalline cetirizine dihydrochloride salt, with the remainder being the amorphous form of the salt. Then, X-ray diffraction patterns are obtained for each artificial mixture using standard X-ray diffraction techniques. Slight variations in peak positions, if any, may be accounted for by adjusting the location of the peak to be measured. The intensities of the 100% peak(s) for each of the artificial mixtures are then plotted against the known weight percentages of the crystalline form of the salt. The resulting plot is a calibration curve that allows determination of the amount of crystalline cetirizine dihydrochloride salt in an unknown sample. For the unknown mixture of crystalline and amorphous cetirizine dihydrochloride salt, the intensities of the 100% peak(s) in the mixture, relative to an intensity of this peak in a calibration mixture, may be used to determine the percentage of the crystalline form in the composition, with the remainder determined to be the amorphous material.

The invention also provides a process for preparation of amorphous cetirizine dihydrochloride salt. The starting material for preparation of amorphous cetirizine dihydrochloride salt may be cetirizine free base or salt other than dihydrochloride. In this case, the starting material is suspended or dissolved in a solvent carrier and a suitable amount of hydrochloric acid is added to convert the starting material to the dihydrochloride salt. If the starting material is dihydrochloride salt of cetirizine (e.g., crystalline or oil form), addition of hydrochloric acid may be unnecessary. The solvent carrier may be a mixture of water with an organic solvent. If the starting material is cetirizine free base, it may be suspended in the water-based solvent carrier and dissolves as the dihydrochloride salt is formed upon addition of the hydrochloric acid. Then, the solvent is removed, for example, by evaporation under vacuum or otherwise to obtain a residue of dihydrochloric salt, which is then triturated with hydrocarbon solvent.

In one specific embodiment, the amorphous form of cetirizine dihydrochloride may be prepared, for example, by

(i) providing cetirizine free base or salt thereof in a solvent carrier,

(ii) treating the cetirizine in said carrier with hydrochloric acid;

(iii) removing the solvent carrier to obtain a residue;

(iv) adding water immiscible aromatic or aliphatic or alicyclic hydrocarbon solvents such as toluene, xylene, cyclohexane or heptane, preferably cyclohexane to said residue thereby said amorphous form of cetirizine dihydrochloride separates as a solid mass;

(v) filtering the compound;

(vi) drying the compound to isolate the desired amorphous form of cetirizine dihydrochloride.

Examples of solvent carriers include, but are not limited to, water; a ketone solvent, such as acetone, methyl ethyl ketone, 2-pentanone or a mixture thereof; a mixture of water and water-miscible solvents like C₁-C₅ straight or branched chain alcoholic solvents (e.g., methanol, ethanol, n-propanol, isopropanol, 2-butanol, n-butanol, n-pentanol or 2-pentanol); a nitrile solvent, such as acetonitrile or propionitrile; and water immiscible aromatic or aliphatic or alicyclic hydrocarbon solvent, such as toluene, cyclohexane or heptane. Acetone, isopropanol, acetonitrile, and toluene are preferred.

The amorphous and crystalline forms of dihydrochloride salt of cetirizine described herein are thermally stable and may be used as an active ingredient in pharmaceutical formulations. The pharmaceutical compositions of the invention may contain the amorphous or crystalline form of dihydrochloride salt of cetirizine as the active ingredient, and one or more pharmaceutically acceptable excipients. Suitable pharmaceutically acceptable excipients include starches, sugars, celluloses, such as microcrystalline cellulose, hydroxypropyl cellulose, and hydroxypropylmethyl cellulose, diluents, granulating agents, lubricants, binders, disintegrating agents, and the like.

The amorphous form of the composition comprising cetirizine dihydrochloride salt has a moisture content which varies from 0.3 to 12.0% by KF method. Typically, the moisture content of the substance is around 1.5 to 7.5% by KF method. The moisture content of present inventive substance was measured on Mettler DL-35 instrument using Karl-Fischer reagent.

Generally, the pharmaceutical compositions of the present invention are prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed. Examples of formulations suitable for the amorphous form of cetirizine dihydrochloride salt of the invention are disclosed in U.S. Pat. Nos. 6,245,353 and 5,698,558, the disclosures of which are incorporated herein by reference in their entirety.

The more preferred oral solid preparation is a tablet. A tablet may be prepared by direct compression, wet granulation, or molding, of the amorphous form of cetirizine dihydrochloride salt with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent, are suitable in the case of oral solid dosage forms (e.g., powders, capsules, and tablets). If desired, tablets may be coated by standard techniques. The amorphous form of cetirizine dihydrochloride salt described herein may be formulated into typical disintegrating tablet, or into a controlled or extended release dosage forms. Examples of suitable controlled release formulation vehicles are disclosed in U.S. Pat. Nos. 3,845,770; 3,916,899; 3,536,809; 3,598,123; and 4,008,719, the disclosures of which are hereby incorporated by reference in their entirety. U.S. Pat. No. 5,698,558, incorporated by reference in its entirety, discloses a method of utilizing cetirizine, or a pharmaceutically acceptable salt thereof, substantially free of its (+) stereoisomer.

Preferably, each tablet contains from about 2 mg to about 10 mg of the amorphous form of dihydrochloride salt of cetirizine, and each cachet or capsule contains from about 2 mg to about 10 mg of the amorphous form of dihydrochloride salt of cetirizine. Most preferably, the tablet contains about 2 mg, about 5 mg or about 10 mg of the amorphous form of dihydrochloride salt of cetirizine for oral administration.

The prophylactic or therapeutic dose of the amorphous form of the dihydrochloride salt of cetirizine will vary with the severity of the condition to be treated and the route of administration. The dose and perhaps the dose frequency will also vary according to the age, body weight and response of the individual patient. In general, the total daily dose range for the either the crystalline or amorphous form of cetirizine dihydrochloride salt is from about 1.0 mg to about 25 mg. Preferably, a daily dose range should be about 2.0 mg to about 20 mg in single or divided doses; most preferably, the dose range is from about 5 mg to about 10 mg per day. It is known that children and elderly patients, as well as those with impaired renal or hepatic function, should receive low doses, at least initially.

The term “prophylactically or therapeutically effective amount” refers to the above-described dosage amounts and dose frequency schedules. Any suitable route of administration may be employed. For example, oral, rectal, parenteral (subcutaneous, intramuscular, intravenous), and transdermal, and like forms of administration may be suitable. Oral route of administration is preferred.

Hence, the present invention is directed to provide both crystalline and amorphous forms of dihydrochloride salts of cetirizine. The processes described herein are simple, eco-friendly and commercially viable.

### EXAMPLES

The invention is further defined by reference to the following examples describing in detail the preparation of the compound and the compositions of the present invention, as well as their utility. It will be apparent to those skilled in the art, that many modifications, both to materials, and methods, may be practiced without departing from the purpose and interest of this invention.

### Reference Example

### Preparation of Crude Levorotatory Cetirizine

Levorotatory [2-[4-[(4-Chlorophenyl)-phenyl methyl]-1-piperazinyl] ethanol (55 grams) was dissolved in dimethyl formamide (165 ml) and cooled to a temperature of 0-5° C. Potassium hydroxide (28.0 grams) was added to the reaction mixture and maintained for 90 minutes. Sodium monochloroacetate (29.0 grams) was then added and further maintained at a temperature of 0-5° C. for 90 minutes. The temperature of the reaction mixture was raised to 30-35° C. and maintained until the reaction was substantially complete. Water (605 ml) was added to the reaction mixture and the temperature of the reaction mixture was raised to 40-50° C. The reaction mixture was then washed with toluene (4×110 ml). The pH of the aqueous layer was adjusted to 4-4.5 with Hydrochloric acid and extracted with dichloromethane (2×165 ml). The extracted organic layer was first washed with 10% Sodium chloride solution (2×165 ml), and then washed with water (2×165 ml). Carbon (2.7 g) was added to the washed organic layer and heated to reflux temperature. The reaction mixture was filtered and then washed with dichloromethane (55 ml) to separate the layers. The solvent was evaporated off of the reaction solution under vacuum to afford crude levorotatory [2-[4-[(4-Chlorophenyl)-phenyl methyl]-1-piperazinyl] ethoxy] acetic acid (Weight: 60.6 grams).

### Reference Example

### Preparation of Crude Dextrorotatory Cetirizine

Dextrorotatory [2-[4-[(4-Chlorophenyl)-phenyl methyl]-1-piperazinyl] ethanol (105 grams) was dissolved in dimethyl formamide (357 ml) and cooled to a temperature of 0-5° C. Potassium hydroxide (53.3 grams) was added to the reaction mixture and maintained for 90 minutes. Sodium monochloroacetate (55.5 grams) was then added and further maintained at a temperature of 0-5° C. for 90 minutes. The temperature of the reaction mixture was raised to 30-35° C. and maintained until the reaction was substantially complete. Water (1155 ml) was then added to the reaction mixture. The pH of the aqueous layer was adjusted to 9.5 with Hydrochloric acid until the bi-layer mixture separated. The bilayer mixture was further separated and washed with ethyl acetate (280×1+245×2 ml). The pH of the aqueous layer was adjusted to 4-4.5 with Hydrochloric acid and extracted with dichloromethane (385×1+2×245 ml). The extracted organic layer was first washed with 10% Sodium chloride solution (1×200 ml), and then washed with water (200 ml). The solvent was evaporated off of reaction solution under vacuum to afford crude dextrorotatory [2-[4-[(4-Chlorophenyl)-phenyl methyl]-1-piperazinyl] ethoxy] acetic acid (Weight: 123.0 grams).

### Example 1

### Preparation of Crystalline Form I Dextrorotatory Dihydrochloride Salt of Cetirizine

Crude levorotatory [2-[4[4(4-Chlorophenyl)-phenyl methyl]-1-piperazinyl] ethoxy] acetic acid (56.6 grams) was dissolved in acetone (825 ml) at a temperature of 40-50° C. Hydrochloride acid (32.0 ml) was added to the reaction mixture and the reaction solution was stirred to separate the solid. The solid was filtered, washed with acetone (55 ml) and dried at a temperature of 55-60° C. to obtain the crude product of dextrorotatory dihydrochloride salt of rotatory [2-[4[4(4-Chlorophenyl)-phenyl methyl]-1-piperazinyl] ethoxy] acetic acid. (42.0 grams) The crude product (40.0 g) was then washed with aqueous acetone and upon subsequent drying to a constant weight, resulted the crystalline Form-1 of dextrorotatory dihydrochloride salt of rotatory [2-[4[4(4-Chlorophenyl)-phenyl methyl]-1-piperazinyl] ethoxy] acetic acid. (Weight: 33.9 grams, Optical Rotation=(+) 12.5° C.=1% in water at 365 nm).

### Example 2

### Preparation of Crystalline Form I Levorotatory Dihydrochloride Salt of Cetirizine

Dextrorotatory [2-[4 [4(4-Chlorophenyl)-phenyl methyl]-1-piperazinyl] ethoxy] acetic acid (110 grams) was dissolved in acetone (1100 ml) at a temperature of 25-35° C. Carbon (5.5 g) was added to the reaction mixture and the solution was stirred for a period of 15-30 minutes. Hydrochloric acid (64 ml) was added to the solution and the temperature was raised to 45-50° C. and the solution was stirred for a period of 1-2 hours. The reaction mixture was cooled to room temperature and stirred for 1-2 hours to separate the solid. The separated solid was filtered, washed with acetone (550 ml) and subsequently dried at a temperature of 55-60° C. to yield crystalline Form-I Levorotatory dihydrochloride salt of [2-[4[4(4-Chlorophenyl)-phenyl methyl]-1-piperazinyl] ethoxy] acetic acid. (71.4 g) The crude product (70.0 g) was washed with aqueous acetone to yield crystalline Form-1 levorotatory dihydrochloride salt of [2-[4[4(4-Chlorophenyl)-phenyl methyl]-1-piperazinyl] ethoxy] acetic acid. (Weight 58.5 g, Optical Rotation=(−)12.2°, C=1% in water at 365 nm)

### Example 3

### Preparation of Amorphous Dextrorotatory Dihydrochloride Salt of Cetirizine

Crystalline dextrorotatory dihydrochloride salt of [2-[4[4(4-Chlorophenyl)-phenyl methyl]-1-piperazinyl] ethoxy] acetic acid (10 grams) was dissolved in a mixture of water (100 ml) and acetone (40 ml) at room temperature. The reaction mixture was filtered and the solvent was distilled off to dryness at a temperature below 80° C. under vacuum to separate the solid. The solid was dried at a temperature of 80-90° C. to a constant weight to afford the amorphous form of dextrorotatory dihydrochloride salt of cetirizine. (Weight 9.5 grams, M.C. by KF: 1.5%, Optical Rotation=(+) 12.1°, C=1 in water at 365 nm)

### Example 4

Levorotatory cetirizine (10 grams) was dissolved in a mixture of water (40 ml) and acetone (100 ml) at room temperature. Hydrochloric acid (10 ml) was added to the reaction mixture and stirred for 10 to 30 minutes at a temperature of 30-35 ° C. The reaction solution was filtered and the solvent was distilled off to dryness at a temperature below 80° C. Cyclohexane (100 ml) was added to the residual mass and stirred for 30-60 minutes at a temperature of 30-35° C. The product was filtered and then washed with cyclohexane (50 ml). The resulted product was dried at a temperature of 80-85° C. to a constant weight to afford the amorphous form of dextrorotatory dihydrochloride salt of cetirizine. (Weight 9.9 grams)

### Example 5

### Preparation of Amorphous Levorotatory Dihydrochloride Salt of Cetirizine

Crystalline levorotatory dihydrochloride salt of cetirizine (5.0 grams) was dissolved in a mixture of water (50 ml) and acetone (20 ml). The reaction mixture was stirred at a temperature of 25-35° C. until the reaction solution became clear. The reaction solution was filtered and the solvent was distilled off to dryness at a temperature of 50-75° C. under reduced pressure to result the amorphous form of levorotatory cetirizine dihydrochloride. The amorphous form of levorotatory cetirizine dihydrochloride was further dried at a temperature of 65-70° C. to a constant weight to afford the novel amorphous form of levocetirizine dihydrochloride. (Weight: 4.2 grams; M.C. by KF: 5.8%, Optical Rotation=(−) 11.7, C=1 in water at 365 nm)

### Example 6

Dextrocetirizine (5 grams) was dissolved in a mixture of water (20 ml) and acetone (50 ml) at room temperature. Hydrochloric acid (5 ml) was added to the reaction mixture and the solution was stirred for 10 to 30 minutes at a temperature of 30 to 35° C. The reaction solution was then filtered and the solvent was distilled off to dryness at a temperature below 80° C. Cyclohexane (50 ml) was added to the residual mass and the solution was stirred for 30 minutes at a temperature of 30-35° C. The product was filtered and washed with cyclohexane (25 ml) and dried at a temperature of 60-110° C. to a constant weight to afford the amorphous form of Levorotatory dihydrochloride salt of cetirizine. (Weight: 4.7 grams, M.C. by KF: 1.7%)

### Example 8

### Soluble Granules Containing Amorphous Dihydrochloride Salt of Cetirizine

Soluble granules containing amorphous dihydrochloride salt of cetirizine may have the following content:

| Ingredient | Content (mg) |
|---|---|
| Amorphous dihydrochloride salt | 10 |
| of cetirizine | |
| Calcium carbonate | 750 |
| Citric acid | 950 |
| Avicel | 35 |
| Mannitol | 620 |
| Maltodextrin | 16 |
| Aspartame | 4 |
| Aroma | 18 |

### Example 9

### Dispersible Tablet Containing Dihydrochloride Salt of Cetirizine

Dispersible tablet containing amorphous form of dihydrochloride salt of cetirizine may have the following content:

| Ingredient | Content (mg) |
|---|---|
| Amorphous dihydrochloride salt of | 10 |
| Cetirizine | |
| Calcium carbonate | 450 |
| Polyvinylpyrrolidone | 20 |
| Avicel | 12 |
| Mannitol | 450 |
| Maltodextrin | 15 |
| Aspartame | 7 |
| Aroma | 16 |

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. A crystalline form of dextrorotatory dihydrochloride salt of [2-[4-[(4-chlorophenyl)-phenyl methyl]-1-piperazinyl] ethoxy] acetic acid (dextrorotatory dihydrochloride salt of cetirizine).

2. A crystalline form of a dextrorotatory dihydrochloride salt of cetirizine having substantially the same X-ray diffraction pattern as shown in FIG. 1.

3. A crystalline form of dextrorotatory dihydrochloride salt of cetirizine having an X-ray diffraction pattern expressed in terms of 2 theta angles and obtained with a diffractometer equipped with a copper K X-radiation source, wherein said X-ray powder diffraction pattern includes five or more peaks selected from the group consisting of peaks with 2 theta angles of 7.05±0.09, 7.96±0.09, 14.35±0.09, 14.81±0.09, 17.40±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09 and 30.57±0.09.

4. The crystalline form of dextrorotatory dihydrochloride salt of cetirizine of claim 1 that has an endo-endo pattern with identified peaks of about 195° C. and 215° C. in its differential scanning colorimetry thermogram.

5. The crystalline form of dextrorotatory dihydrochloride salt of cetirizine of claim 1 having an infrared spectrum with identifiable peaks at about 3430, 2949, 2376, 1746, 1497, 1320, 1137, 920, 759, 720, 700 and 534 cm⁻¹.

6. A pharmaceutical composition comprising a prophylactically or therapeutically effective amount of the crystalline form of dextrorotatory dihydrochloride salt of cetirizine of claim 1, and one or more pharmaceutically acceptable excipients.

7. The pharmaceutical composition of claim 6, wherein said crystalline form of dextrorotatory dihydrochloride salt of cetirizine has an X-ray diffraction pattern expressed in terms of 2 theta angles and obtained with a copper K X-radiation source, wherein said X-ray powder diffraction pattern includes five or more peaks selected from the group consisting of peaks with 2 theta angles of 7.05±0.09, 7.96±0.09, 14.35±0.09, 14.81±0.09, 17.40±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09, and 30.57±0.09.

8. A process for preparation of a crystalline form of dextrorotatory dihydrochloride salt of cetirizine, said process comprising:
a) providing a solution of dextrorotatory dihydrochloride salt of cetirizine in a ketone-containing solvent;
b) cooling said solution thereby a solid separates; and
c) isolating said solid mass thereby obtaining said crystalline form of dextrorotatory dihydrochloride salt of cetirizine.

9. The process according to claim 8, wherein said ketone-containing solvent comprises water and a ketone selected from the group consisting of acetone, methyl ethyl ketone, dimethyl ketone, 2-pentanone, and mixtures thereof.

10. The process according to claim 9, wherein said ketone-containing solvent comprises water and acetone.

11. The process according to claim 9, which further comprises drying said isolated solid mass at from about 40° C. to about 100° C.

12. The process according to claim 11, wherein said isolated mass is dried at from about 55° C. to about 65° C.

13. A crystalline form of dextrorotatory dihydrochloride salt of cetirizine produced in accordance with the process of claim 8.

14. The crystalline form of dextrorotatory dihydrochloride salt of cetirizine of claim 13 and having an X-ray diffraction pattern expressed in terms of 2 theta angles and obtained with a diffractometer equipped with a diffracted beam curved graphite monochromator using copper K X-radiation source, wherein said X-ray powder diffraction pattern includes five or more peaks selected from the group consisting of peaks with 2 theta angles of 7.05±0.09, 7.96±0.09, 14.35±0.09, 14.81±0.09, 17.40±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09 and 30.57±0.09.

15. A pharmaceutical composition comprising a) a prophylactically or therapeutically effective amount of the crystalline form of dextrorotatory dihydrochloride salt of cetirizine produced by the process of claim 8, and b) one or more pharmaceutically acceptable excipients.

16. A pharmaceutical composition comprising a) a prophylactically or therapeutically effective amount of the crystalline form of dextrorotatory dihydrochloride salt of cetirizine produced by the process of claim 8 and having an X-ray diffraction pattern expressed in terms 2 theta angles and obtained with a diffractometer equipped with a diffracted beam curved graphite monochromator using copper K X-radiation source, wherein said X-ray powder diffraction pattern includes five or more peaks selected from the group consisting of peaks with 2 theta angles of 7.05±0.09, 7.96±0.09, 14.35±0.09, 14.81±0.09, 17.40±0.09, 18.17±0.09, 18.59±0.09, 18.82±0.09, 20.33±0.09, 22.33±0.09, 23.35±0.09, 24.16±0.09, 24.33±0.09, 24.73±0.09, 25.28±0.09, 26.51±0.09, 26.80±0.09, 27.35±0.09, and 30.57±0.09, and b) one or more pharmaceutically acceptable excipients.

17. A crystalline form of levorotatory dihydrochloride salt of [2-[4-[(4-Chlorophenyl)-phenyl methyl]-1-piperazinyl] ethoxy] acetic acid (levorotatory dihydrochloride salt of cetirizine).

18. A crystalline form of a levorotatory dihydrochloride salt of cetirizine having substantially the same X-ray diffraction pattern as shown in FIG. 2.

19. A crystalline form of levorotatory dihydrochloride salt of cetirizine having an X-ray diffraction pattern expressed in terms of 2 theta angles and obtained with a diffractometer equipped with a copper K X-radiation source, wherein said X-ray powder diffraction pattern includes five or more peaks selected from the group consisting of peaks with 2 theta angles of 7.10±0.09, 8.02±0.09, 14.41±0.09, .14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

20. The crystalline form of levorotatory dihydrochloride salt of cetirizine of claim 17 that has an endo-endo pattern with identified peaks of about 195° C. and 215° C. in its differential scanning colorimetry thermogram.

21. The crystalline form of levorotatory dihydrochloride salt of cetirizine of claim 17 having an infrared spectrum with identifiable peaks at about 3430, 2949, 2376, 1746, 1497, 1320, 1137, 920, 759, 720, 700 and 534 cm⁻¹.

22. A pharmaceutical composition comprising a prophylactically or therapeutically effective amount of the crystalline form of levorotatory dihydrochloride salt of cetirizine of claim 17, and one or more pharmaceutically acceptable excipients.

23. The pharmaceutical composition of claim 22, wherein said crystalline form of levorotatory dihydrochloride salt of cetirizine has an X-ray diffraction pattern expressed in terms of 2 theta angles and obtained with a copper K X-radiation source, wherein said X-ray powder diffraction pattern includes five or more peaks selected from the group consisting of peaks with 2 theta angles of 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

24. A process for preparation of a crystalline form of levorotatory dihydrochloride salt of cetirizine, said process comprising:
a) providing a solution of levorotatory dihydrochloride salt of cetirizine in a ketone-containing solvent;
b) cooling said solution thereby a solid separates; and
c) isolating said solid mass thereby obtaining said crystalline form of levorotatory dihydrochloride salt of cetirizine.

25. The process according to claim 24, wherein said ketone-containing solvent comprises water and a ketone selected from the group consisting of acetone, methyl ethyl ketone, dimethyl ketone, 2-pentanone, and mixtures thereof.

26. The process according to claim 25, wherein said ketone-containing solvent comprises water and acetone.

27. The process according to claim 24, which further comprises drying said isolated solid mass at from about 40° C. to about 100° C.

28. The process according to claim 24, wherein said isolated solid mass is dried at from about 55° C. to about 65° C.

29. The crystalline form of levorotatory dihydrochloride salt of cetirizine produced in accordance with a process of claim 24.

30. The crystalline form of levorotatory dihydrochloride salt of cetirizine of claim 29 and having an X-ray diffraction pattern expressed in terms of 2 theta angles and obtained with a diffractometer equipped with a diffracted beam curved graphite monochromator using copper K X-radiation source, wherein said X-ray powder diffraction pattern includes five or more peaks selected from the group consisting of peaks with 2 theta angles of 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

31. A pharmaceutical composition comprising a) a prophylactically or therapeutically effective amount of the crystalline form of levorotatory dihydrochloride salt of cetirizine produced by the process of claim 24, and b) one or more pharmaceutically acceptable excipients.

32. A pharmaceutical composition comprising a) a prophylactically or therapeutically effective amount of the crystalline form of levorotatory dihydrochloride salt of cetirizine produced by the process of claim 24 and having an X-ray diffraction pattern expressed in terms of 2 theta angles and obtained with a diffractometer equipped with a diffracted beam curved graphite monochromator using copper K X-radiation source, wherein said X-ray powder diffraction pattern includes five or more peaks selected from the group consisting of peaks with 2 theta angles of 7.10±0.09, 8.02±0.09, 14.41±0.09, 14.87±0.09, 17.48±0.09, 18.24±0.09, 18.65±0.09, 18.86±0.09, 22.39±0.09, 23.42±0.09, 24.21±0.09, 24.36±0.09, 24.81±0.09, 25.31±0.09, 26.60±0.09 and 29.28±0.09.

33. The pharmaceutical composition of claim 16 or 32, which is a solid dosage form for oral administration.

34. The pharmaceutical composition of claim 33, wherein said solid dosage form is a tablet.

35. An amorphous form of dextrorotatory dihydrochloride salt of cetirizine.

36. An amorphous form of dextrorotatory dihydrochloride salt of cetirizine, which is substantially free of crystalline forms of dextrorotatory dihydrochloride salt of cetirizine.

37. An amorphous form of dextrorotatory dihydrochloride salt of cetirizine characterized by an X-ray powder diffraction pattern substantially in accordance with FIG. (3).

38. A pharmaceutical composition comprising a prophylactically or therapeutically effective amount of an amorphous form of a dextrorotatory dihydrochloride salt of cetirizine, and one or more pharmaceutically acceptable excipients.

39. The pharmaceutical composition of claim 38, which is substantially free of crystalline forms of dextrorotatory dihydrochloride salt of cetirizine.

40. A composition comprising dextrorotatory dihydrochloride salt of cetirizine as a solid, wherein at least 80% by weight of said dextrorotatory dihydrochloride salt of cetirizine is in an amorphous form.

41. The composition of claim 40, wherein at least 90% of said solid dextrorotatory dihydrochloride salt of cetirizine is in an amorphous form.

42. The composition of claim 40, wherein at least 95% of said solid dextrorotatory dihydrochloride salt of cetirizine is in an amorphous form.

43. The composition of claim 40, wherein at least 99% of said solid dextrorotatory dihydrochloride salt of cetirizine is in an amorphous form.

44. The composition of claim 40, which is substantially free of the crystalline forms of dextrorotatory dihydrochloride salt of cetirizine.

45. The composition of claim 40, wherein at most 1% of said solid dextrorotatory dihydrochloride salt of cetirizine is in a crystalline form.

46. The composition of claim 40, wherein at most 5% of said solid dextrorotatory dihydrochloride salt of cetirizine is in a crystalline form.

47. The composition of claim 40 having a moisture content ranging from about 0.3% to about 12% by KF method.

48. The composition of claim 40 having a moisture content ranging from about 1.5% to about 7.5% by KF method.

49. A process for the preparation of an amorphous form of dextrorotatory dihydrochloride salt of cetirizine, said process comprising:
a) dissolving a dextrorotatory dihydrochloride salt of cetirizine in a ketone containing solvent at about 25° C. to about 40° C.;
b) distilling said ketone-containing solvent below about 80° C. under reduced pressure to obtain a residue and
c) drying said residue at a temperature below about 100° C. to obtain the amorphous form of dextrorotatory dihydrochloride salt of cetirizine.

50. The process according to claim 49, wherein said ketone-containing solvent comprises water and a ketone selected from the group consisting of acetone, methyl ethyl ketone, acetone, methyl ethyl ketone, dimethyl ketone, 2-pentanone, and mixtures thereof.

51. The process according to claim 49, wherein said ketone-containing solvent comprises water and acetone.

52. The process according to claim 49, wherein said dissolution step is carried out at from about 25° C. to about 35° C.

53. The process according to claim 49, wherein said drying step is conducted at about 40° C. to about 100° C.

54. The process according to claim 53, wherein said drying step is conducted at about 80° C. to about 90° C.

55. The process according to claim 49, wherein said dextrorotatory dihydrochloride salt of cetirizine of step a) is crystalline.

56. The amorphous form of dextrorotatory dihydrochloride salt of cetirizine produced in accordance with the process of claim 49.

57. A pharmaceutical composition comprising i) a prophylactically or therapeutically effective amount of dextrorotatory dihydrochloride salt of cetirizine in a solid form produced by the process of claim 49, and ii) one or more pharmaceutically acceptable excipients.

58. The composition of claim 57, wherein said pharmaceutical composition is a solid dosage form for oral administration.

59. The composition of claim 58, wherein said solid dosage form is a tablet.

60. The composition of claim 57, having a moisture content ranging from about 0.3% to about 12% by KF method.

61. The composition of claim 57, having a moisture content ranging from about 1.5% to about 7.5% by KF method.

62. An amorphous form of levorotatory dihydrochloride salt of cetirizine.

63. An amorphous form of levorotatory dihydrochloride salt of cetirizine, which is substantially free of crystalline forms of levorotatory dihydrochloride salt of cetirizine.

64. An amorphous form of levorotatory dihydrochloride salt of cetirizine characterized by an X-ray powder diffraction pattern substantially in accordance with FIG. (4).

65. A pharmaceutical composition comprising a prophylactically or therapeutically effective amount of an amorphous form of a levorotatory dihydrochloride salt of cetirizine and one or more pharmaceutically acceptable excipients.

66. The pharmaceutical composition of claim 65, which is substantially free of crystalline levorotatory dihydrochloride salt of cetirizine.

67. A composition comprising levorotatory dihydrochloride salt of cetirizine as a solid, wherein at least 80% by weight of said levorotatory dihydrochloride salt of cetirizine is in an amorphous form.

68. The composition of claim 67, wherein at least 90% of said solid levorotatory dihydrochloride salt of cetirizine is in an amorphous form.

69. The composition of claim 67, wherein at least 95% of said solid levorotatory dihydrochloride salt of cetirizine is in an amorphous form.

70. The composition of claim 67, wherein at least 99% of said solid levorotatory dihydrochloride salt of cetirizine is in an amorphous form.

71. The composition of claim 67, which is substantially free of the crystalline forms of levorotatory dihydrochloride salt of cetirizine.

72. The composition of claim 67, wherein at most 1% of said solid levorotatory dihydrochloride salt of cetirizine is in a crystalline form.

73. The composition of claim 67, wherein at most 5% of said solid levorotatory dihydrochloride salt of cetirizine is in a crystalline form.

74. The composition of claim 67 having a moisture content ranging from about 0.3% to about 12% by KF method.

75. The composition of claim 67 having a moisture content ranging from about 1.5% to about 7.5% by KF method.

76. A process for the preparation of an amorphous form of levorotatory dihydrochloride salt of cetirizine, said process comprising:
a) dissolving a levorotatory dihydrochloride salt of cetirizine in a ketone-containing solvent at about 25° C. to about 40° C.;
b) distilling said ketone-containing solvent below about 80° C. under reduced pressure to obtain a residue; and
c) drying said residue at a temperature below about 100 C to obtain the amorphous form of levorotatory dihydrochloride salt of cetirizine.

77. The process according to claim 76, wherein said ketone-containing solvent comprises water and a ketone selected from the group consisting of acetone, methyl ethyl ketone, acetone, methyl ethyl ketone, dimethyl ketone, 2-pentanone, and mixtures thereof.

78. The process according to claim 76, wherein said ketone-containing solvent comprises water and acetone.

79. The process according to claim 76, wherein said dissolution step is carried out at from about 25° C. to about 35° C.

80. The process according to claim 76, wherein said drying step is conducted at about 40° C. to about 100° C.

81. The process according to claim 76, wherein said drying step is conducted at about 80° C. to about 90° C.

82. The process according to claim 76, wherein said levorotatory dihydrochloride sat of cetirizine is crystalline.

83. The amorphous form of levorotatory dihydrochloride salt of cetirizine produced in accordance with the process of claim 76.

84. A pharmaceutical composition comprising i) a prophylactically or therapeutically effective amount of levorotatory dihydrochloride salt of cetirizine in a solid form produced by the process of claim 76, and ii) one or more pharmaceutically acceptable excipients.

85. The composition of claim 84, wherein said pharmaceutical composition is a solid dosage form for oral administration.

86. The composition of claim 85, wherein said solid dosage form is a tablet.
